(19) United States
(12) Patent Application Publication
(10) Pub. No.: US 2007/0213669 A1
(43) Pub. Date: Sep. 13, 2007

(54) Flexible sleeve, adjustable contact surface, and fluid contact monitor for catheter

(75) Inventors: Alan D. Eskuri, Hanover, MN (US); Christopher Johnson, Brooklyn Center, MN (US); Chad Harris, Albertville, MN (US); Matthew L. Hawk, Otsego, MN (US); Grace Kim, Minneapolis, MN (US)

Correspondence Address: KENYON & KENYON LLP, 1500 K STREET N.W., SUITE 700, WASHINGTON, DC, 20005, US

(73) Assignee: Boston Scientific Scimed, Inc.

(21) Appl. No.: 11/368,540

(22) Filed: Mar. 7, 2006

(51) Int. Cl. A61M 5/178

(52) U.S. Cl. 604/164.01

(57) ABSTRACT

A catheter is provided for the injection of therapeutic agents at a target site within a patient's body. The catheter includes a first elongated shaft having a distal end and a proximal end and a lumen extending therebetween. The catheter also includes a second elongated shaft in the form of a needle slidingly disposed in the first elongated shaft, the needle having a distal end and a proximal and a lumen extending therebetween. The catheter may include a flexible sleeve, wherein the flexible sleeve compresses along the inside of a curve and elongates along the outside of a curve. The catheter may additionally or alternatively include an adjustable distal tissue contact surface, wherein the position of the distal tissue contact surface may be adjusted with respect to a stop surface for stopping the advancement of a needle. The catheter may additionally or alternatively include a fluid pressure contact monitor.

### FIELD OF THE INVENTION

The present invention relates to medical catheters, for example injection catheters, and to methods of using such catheters.

### BACKGROUND

Medical catheters are used for a number of minimally invasive medical procedures. For example, catheters may be used to guide medical instruments to a target site to perform a surgical procedure, such as tissue rescission, ablation of obstructive deposits or myocardial revascularization. Catheters may also be used to deliver implantable medical devices, such as lumen-reinforcing or drug-eluting stents, to an implantation site within the body. Catheters may also be used to deliver therapeutic agents to target tissue. One example of an injection catheter for a particular application is a myocardial injection catheter, having a needle used to deliver therapeutic agents, for example cell and viral therapeutic agents, to the myocardial wall to stimulate myocardial angiogenesis and myocardial tissue regeneration.

One issue with injection catheters is making sure that the injection needle penetrates the target tissue to a sufficient depth. If the depth of injection of the needle causes the needle tip to extend through the tissue (e.g., the ventricular wall), or if the depth of injection of the needle does not extend sufficiently into the tissue, the therapeutic agents will not be delivered to the desired location, and thus the effectiveness of the procedure will be compromised.

One difficulty in obtaining the correct depth of injection is the loss or gain in needle length relative to the catheter shaft or tube due to the bending and curving of the catheter to reach the desired tissue site. For example, when a catheter is inserted into and moved through a body, a needle that is disposed within the catheter tube will be subjected to similar movements and bends as the catheter tube. However, for a variety of reasons, for example friction, the amount of space between the inner surface of the catheter tube and the needle, and different levels of flexibility, the distal end of the needle may not remain in the same position relative to the distal end of the catheter tube when the catheter is in a curved position as when it is in a straight position. In the case where the needle is not as flexible as the catheter tube and the catheter has been contorted to have numerous curves, the needle may extend too far distally or even past the end of the catheter because it has taken a “path of least resistance” to short-cut through the curves in the catheter tube. Thus, when the needle is advanced distally for injection, it may extend farther than desired, causing the depth of the injection to be too deep.

Conversely, bending and curving may result in the tip of the needle not being extended distally enough within the catheter tube. For example, certain catheter designs, such as “Stiletto” catheters marketed by Boston Scientific Corp., incorporate a coil in the catheter tube to provide flexibility with improved kink resistance as compared to polymer extrusions. However, during bending, the windings of the coil may separate at the outside of the curve, and the separation of the windings of the coil causes the length of the coil along the centerline to increase. This lengthens the coil relative to the needle, resulting in the needle tip being located too far proximally with respect to the distal end of the catheter. As a result, during deployment the needle may not be advanced far enough, resulting in a reduced depth of injection or even no injection. For example, if the depth of injection is to be 2 mm and the inner needle has receded 1.5 mm from its at rest position near the distal end of the catheter, then the actual depth of the injection will only be 0.5 mm.

Loss or gain in needle length relative to the distal end of the catheter may also occur due to applied longitudinal force. For example, in the case of a catheter incorporating a coil, the coil may compress when longitudinal force is applied to the catheter. Thus, the coil may shorten in longitudinal length, changing the relative positioning of the distal end of the catheter with respect to the distal end of the needle. Even when the windings of the coil are tightly wound, such loss of length may still occur because the longitudinal forces may cause offset of the windings of the coil.

The issue of accurate injection depth is compounded by differences in tissue. For example, with respect to the example of myocardial injection, not all patients have ventricular walls of equal thickness, which makes it difficult to treat all patients with a needle having a single depth. Similarly, there is a wide range of wall thicknesses even within a single patient's heart.

The issues regarding variable injection depths and differences in length between a catheter tube and needle may arise in various types of injection catheters, not limited to myocardial injection catheters. Certain approaches to resolving these issues are presented in U.S. patent application Ser. No. 10/781,775, filed Feb. 20, 2004, which is hereby incorporated herein by reference. Certain embodiments of the inventions described herein are directed to additional and alternative approaches to resolving these issues.

Another issue with respect to catheters, and particularly with respect to injection catheters, is enabling the operator of the device to know when the device is in the desired position. For example, with an injection catheter, it is desirable for an operator to know when the distal end of the catheter has sufficiently contacted tissue at the injection site, so that the operator may then deploy the needle. Certain approaches to resolving this issue are presented in U.S. patent application Ser. No. 11/037,154, filed Jan. 19, 2005, which is hereby incorporated herein by reference. Certain embodiments of the inventions described herein are directed to additional and alternative approaches to resolving this issue.

### SUMMARY OF THE INVENTION

Certain embodiments of the invention are directed to catheter designs that provide good flexibility without significant changes in length. For example, an injection catheter may have a longitudinally flexible sleeve through which the needle passes. The flexible sleeve is flexible due to openings or slots in the sleeve. The flexible sleeve does not exhibit significant compression when force is applied, and the longitudinal length of the flexible sleeve as measured along the central axis of the flexible sleeve does not exhibit significant changes in length when the sleeve is bent. In this manner, the length of the needle relative to sleeve does not significantly change, thereby making the amount of needle extension beyond the end of the catheter more reliable. Certain embodiments of the invention are directed to methods of using such catheters.

A flexible sleeve for use in an injection catheter in accordance with the invention may be similar in geometry to torqueable tips as shown in U.S. Patent Application No. 2003/0009208 A1 to Snyder et al. and U.S. Pat. No. 6,428,489 to Jacobsen et al., the disclosures of which are hereby incorporated by reference herein. In accordance with the present invention, a flexible sleeve of such a design is used to resist length change and thus to address issues that exist with respect to length changes in prior art injection catheter designs.

Certain embodiments of the invention are directed to catheter designs with an adjustable distal contact surface for controlling the depth of needle injection. For example, an injection catheter may have an adjustable hood that can be moved longitudinally with respect to a stop surface in the catheter. The stop surface limits the distance the needle can be advanced, effectively providing a know distance of needle extension beyond the stop surface. By adjusting the hood with respect to the stop surface, the depth of needle injection can be adjusted. Certain embodiments of the invention are directed to methods of using a catheter with an adjustable distal contact surface.

Certain embodiments of the invention are directed to catheter designs with a fluid pressure sensor at the distal end of the catheter. The fluid pressure sensor senses when the distal end of the catheter is in sufficient contact with the target tissue. Certain embodiments of the invention are directed to methods of using a catheter with a fluid pressure sensor.

Other aspects of embodiments of the invention are set forth in the appended claims.

### BRIEF DESCRIPTION OF THE DRAWINGS

FIG. 1 shows a prior art catheter having a coil in the catheter shaft or tube.

FIG. 2 shows an enlarged view of the distal end of the catheter of FIG. 1.

FIG. 3 shows a first embodiment of a longitudinally flexible sleeve for a catheter.

FIG. 4 shows a second embodiment of a longitudinally flexible sleeve for a catheter.

FIG. 5 shows a third embodiment of a longitudinally flexible sleeve for a catheter.

FIG. 6 shows the distal end of a catheter with a first embodiment of a hood with a needle stop.

FIG. 7 shows the distal end of a catheter with a second embodiment of a hood with a needle stop, wherein the position of the hood is adjustable.

FIG. 8 shows the distal end of a catheter with a fluid pressure tissue contact monitor.

### DETAILED DESCRIPTION

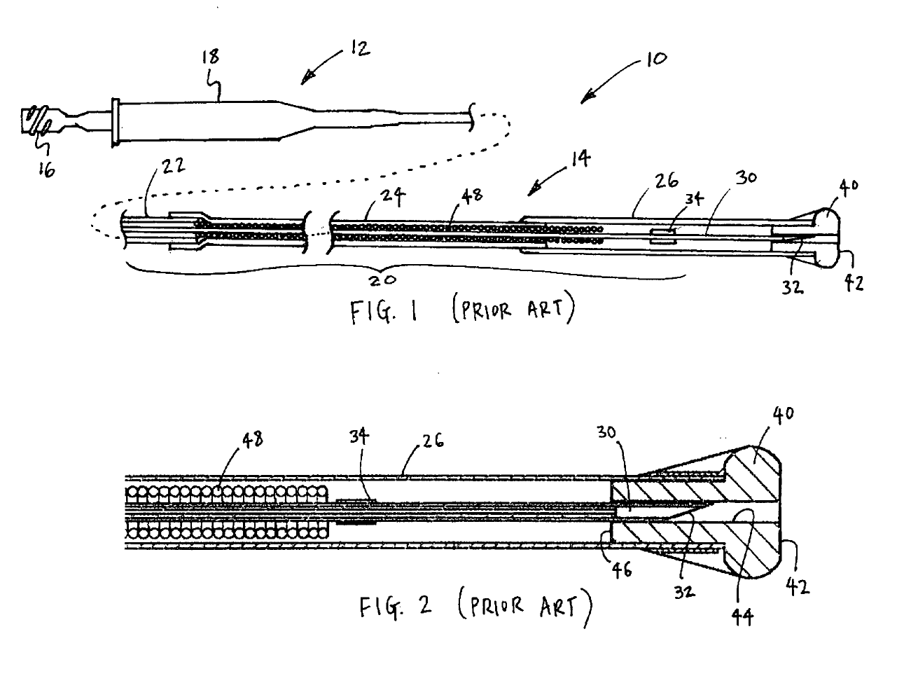

FIG. 1 shows a prior art catheter 10. A proximal section 12 of the catheter is illustrated schematically, and a distal section 14 of the catheter is illustrated in a slightly enlarged and cross-sectional view. FIG. 2 shows as further enlarged and cross-sectional view of the distal-most portion of the catheter 10. The catheter 10 illustrated in FIGS. 1 and 2 is an injection catheter. As would be understood by persons of ordinary skill in the art, the catheter 10 is similar to the “Stiletto” catheters manufactured by Boston Scientific Corp.

As would be understood by persons of ordinary skill in the art, the proximal section 12 of catheter 10 has a hub 16 and manifold 18. The catheter 10 includes an elongated outer catheter shaft or tube 20 which may be constructed in sections. For example, the catheter tube 20 may have a proximal section 22, a first distal section 24, and a second distal section 26. The sections of the catheter tube 20 may be of any suitable construction. For example, the proximal section 22 may be a co-braided structure.

A needle 30 having a needle lumen for therapeutic injection extends through the catheter tube 20 from a proximal end of the catheter 10 to the distal end of the catheter 10. As shown, the needle 30 has a beveled tip 32 to facilitate penetration into target tissue to deliver an injection.

In the illustrated catheter 10, a hood 40 is located at the distal end of the catheter tube 20. The hood provides a contact surface 42 for contacting the target tissue. The distal end of the needle 30 extends through a hood lumen 44 in the hood 40.

The catheter 10 has a coil 48 extending along at least a portion of the catheter tube 20. For example, the coil 48 may extend over much or all of the distal section 14 of the catheter 10. By way of example only, the catheter tube 20 may be approximately 145 cm in length, and the coil 48 may extend over most of the distal 33 cm of that length. Many other lengths are of course possible.

As would be understood by persons of ordinary skill in the art, the coil 48 in the catheter tube 20 provides flexibility to the catheter 10 with improved kink resistance as compared to polymer extrusions. That is, when a polymer tube is subjected to compression forces, particularly when on a bend, it is susceptible to kinking. One way to reduce the kinking tendency is to provide a stiffer polymer tube; however, that reduces flexibility. Flexibility is important so that the catheter can be tracked down tortuous vessels to reach a target site.

While the coil 48 provides both flexibility and improved kink resistance as compared to polymer extrusions, during bending the windings of the coil 48 may separate at the outside of the curve. The separation of the windings of the coil 48 causes the length of the coil 48 along the centerline to increase. This lengthens the coil relative to the needle 30, resulting in the needle tip 32 being located too far proximally with respect to the distal end of the catheter. As a result, during deployment the needle 30 may not be advanced far enough, resulting in a reduced depth of injection or even no injection.

One contributing factor to this loss of length is the fact that the windings of the coil 48 are tight, with adjacent windings abutting or very close to one another, so that the coil can resist longitudinal compression. Thus, when the coil 48 is bent, the windings on the inside of the curve have essentially no room to move closer to one another. Because of this, all or nearly all of the change in length between the inside and outside of the curve that must occur for bending occurs because of lengthening on the outside of the coil 48 rather than compression on the inside of the coil 48. This leads to the lengthening effect at the centerline.

Even when the windings of the coil 48 are tightly wound, a loss of length may still occur under longitudinal forces because the longitudinal forces may cause offset of the windings of the coil. That is, the possibility of this relative movement makes the coil 48 somewhat unstable. In addition, when the coil 48 is bent and subject to longitudinal forces, there is an increased susceptibility of the windings to movement, either through moving the windings together on the outside of the curve or through offset of the coils.

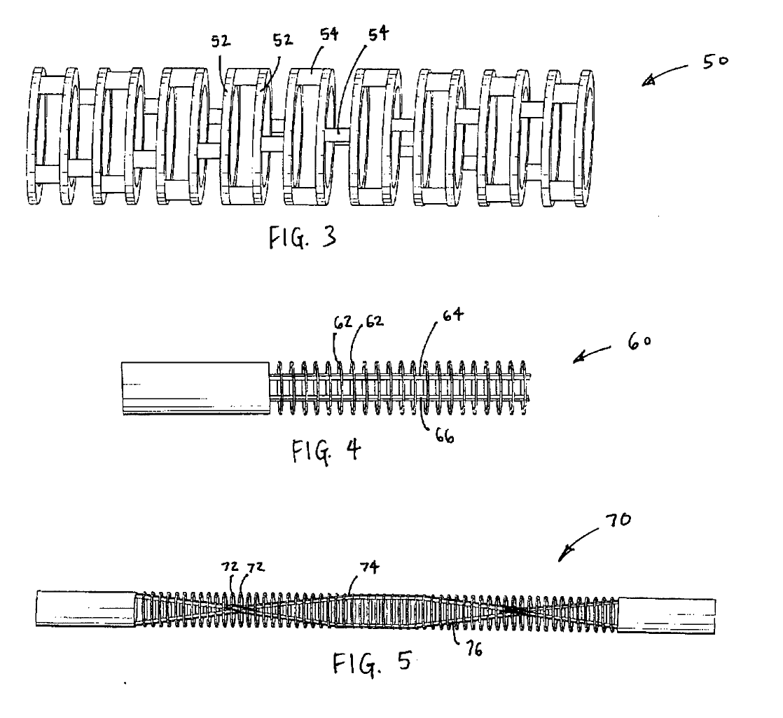

FIGS. 3, 4 and 5 illustrate embodiments of the invention, each showing a longitudinally flexible sleeve for a catheter. The longitudinally flexible sleeve as shown in these figures may be used in place of the coil in a catheter as shown in FIG. 1. In all other respects, the catheter may be the same. FIG. 3 shows a section of a longitudinally flexible sleeve 50. FIG. 4 shows an end section of a longitudinally flexible sleeve 60. FIG. 5 shows a longitudinally flexible sleeve 70.

Longitudinally flexible sleeve 50 shown in FIG. 3 comprises a series of rings 52 connected by straight bar connectors 54. In the places where adjacent rings 52 are not connected to one another, some relative longitudinal movement between the rings 52 can occur, allowing the sleeve 50 to bend. Also, because the sleeve 50 on the inside of a curve can shorten in length while the sleeve 50 on the outside of a curve can increase in length, the centerline can remain at a relatively constant length. Thus, the sleeve 50 can keep a relatively constant position relative to a needle in the catheter.

Longitudinally flexible sleeve 60 shown in FIG. 4 shows an alternative embodiment designed so that the sleeve can better resist longitudinal compression. In sleeve 60, two long connectors 64 and 66 can be used to connect a series of rings 62. Each of the long connectors 64 and 66 extends along a line parallel to the longitudinal axis of the sleeve 60. In this manner, when the sleeve 60 is subjected to longitudinal compressive forces, the two long connectors 64 and 66 resist longitudinal compression, thereby maintaining the length of the sleeve 60. In a similar embodiment, the rings 62 could be connected by a one or more series of straight bar connectors that are lined up along a line parallel to the longitudinal axis of the sleeve, as opposed to staggered as shown in FIG. 5. Lining up the connectors allows the sleeve to resist compressive forces.

Longitudinally flexible sleeve 70 shown in FIG. 5 shows another alternative embodiment designed so that the sleeve 70 can resist longitudinal compression and so that the sleeve can be flexible in all directions. In sleeve 70, two long connectors 74 and 76 can be used to connect a series of rings 72; in this embodiment the long connectors 74 and 76 extend in a helical direction along the length of the sleeve. In this manner, when the sleeve 70 is subjected to longitudinal compressive forces, the two long connectors 74 and 76 resist longitudinal compression, thereby maintaining the length of the sleeve. In addition, because the long connectors 74 and 76 extend in a helical pattern, they do not prevent bending along any one line, such that the sleeve 70 can be bent in any direction. In a similar embodiment, the rings 72 could be connected by a one or more series of bar connectors that are lined up to form a helix down the length of the sleeve. Again, lining up the connectors in this manner allows the sleeve to resist compressive forces.

A longitudinal sleeve as described can be used to surround a needle in an injection catheter. For example, as mentioned above, a longitudinally flexible sleeve as shown and described may be used in place of the coil in a catheter as shown in FIG. 1. As with sleeve 50, in both sleeve 60 and 70, in the places where adjacent rings are not connected to one another, some relative longitudinal movement between them can occur, allowing the sleeve to bend. Also, because the sleeve on the inside of the curve can shorten in length while the sleeve on the outside of the curve increases in length, the centerline can remain at a relatively constant length. Thus, the sleeve can keep a relatively constant position relative to a needle in the catheter.

Other embodiments of flexible sleeves are possible within the scope of the invention. In general, the sleeve allows flexibility while generally retaining centerline length and avoiding significant longitudinal compression.

In accordance with the invention, the material characteristics of the catheter may be selected for the catheter tube and needle to compensate for compression and/or tensile forces. The catheter tube may be subjected to compression forces during advancement of the catheter as well as when the distal end of the catheter is pushed up against tissue, like the heart wall. Similarly, the needle may be subjected to compression forces when deployed into tissue. Tensile forces can be experienced when the catheter is in a bend.

In accordance with the invention, materials may be selected with a desired modulus of elasticity to compensate for the forces and to minimize the length change differences between the catheter tube and needle. The materials may be chosen taking into account both flexibility and compressibility. Possible material choices include nitinol, stainless steel, PEEK, cristamid, the NYLON family of polymers, and nano-composite materials. The tubing for the catheter tube and/or needle may be of any suitable design, including, but not limited to, coiled wires, slotted tubing, e.g., of nitinol, and/or co-braided polymer metal designs.

Referring back to FIGS. 1 and 2, the catheter 10 may also include a needle stop 34 located on the needle 30. The needle stop 34 is sized such that it abuts a stop surface 46 on the hood when advanced. This limits the distance that the needle can extend beyond the distal contact surface 42 of the catheter, to control injection depth.

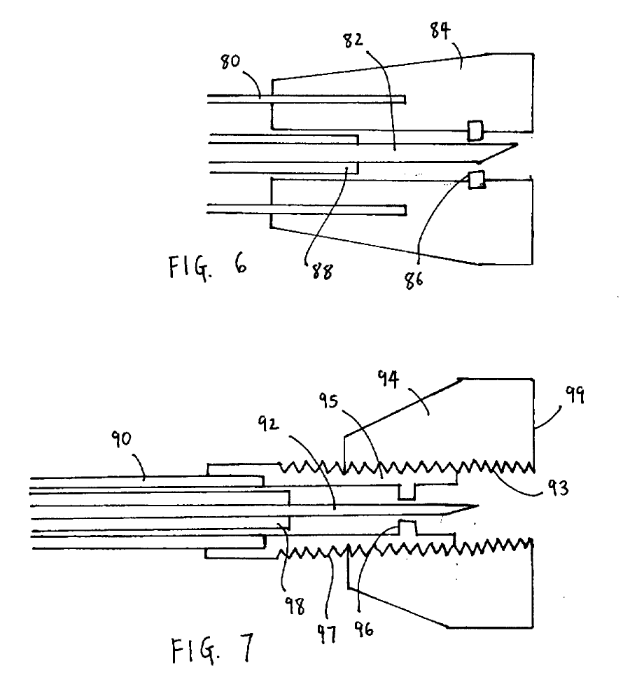

FIG. 6 shows another catheter improvement that can help control needle penetration depth. FIG. 6 shows the distal end of a catheter comprising a catheter tube 80, a needle 82, and a hood 84. On the inside of the hood 84 is a molded stop ring 86. A sleeve 88, for example a PTFE shrink tube, surrounding the needle 82 can be sized such that it contacts the stop ring 86 when the needle is advanced.

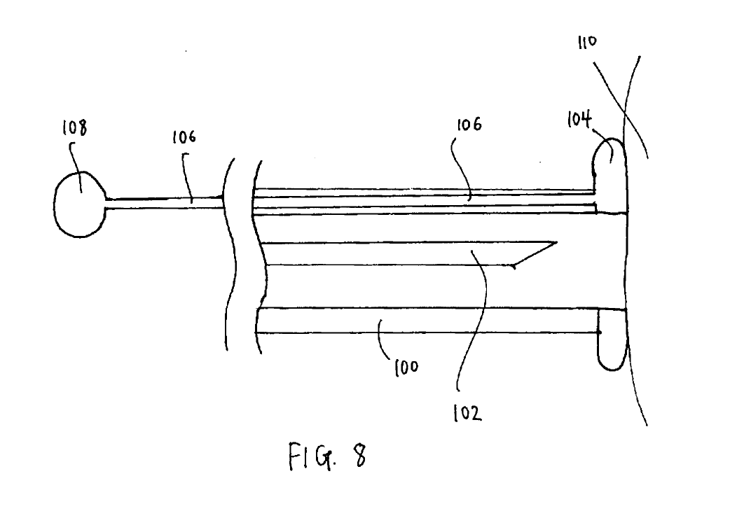

FIG. 7 shows the distal end of a catheter with hood with a needle stop, wherein the position of the hood is adjustable. As shown in FIG. 7, the catheter comprises a catheter tube 90, a needle 92, and a hood 94. The catheter comprises a stop 96 that in this illustration is a part of a molded threaded section 95. A sleeve 98 surrounding the needle 92 can be sized such that it contacts the stop 96 when the needle 92 is advanced.

As shown in FIG. 7, the hood 94 has a threaded surface 93 which engages with a threaded surface 97 of the molded threaded section 95. By rotating the hood 94 with respect to the molded threaded section 95, the hood can be moved distally and proximally with respect to the stop 96. In this manner, the distance from the stop 96 to the distal tissue contact surface 99 of the hood 94 is adjustable. Because the distal tissue contact surface 99 of the hood 94 is the surface that contacts the tissue, adjustment of the hood position adjusts the amount of penetration of the needle.

Adjustment of the needle penetration in the embodiment of FIG. 7 allows the operator to select the amount of needle penetration. The operator may take into account the type of treatment, the patient, the nature of the tissue being injected, etc., in determining the proper setting for needle penetration.

One issue with respect to injection catheters is that it is sometimes difficult for the operator to determine when the catheter is in contact with the tissue to receive the injection. FIG. 8 shows the distal end of a catheter with a fluid pressure tissue contact monitor. As shown in FIG. 8, the catheter comprises a catheter tube 100 and a needle 102. At the distal end of the catheter tube 100 is a fluid pressure tissue contact monitor 104. The monitor 104 may be inflatable from a low profile to a larger profile, similar to an inflatable catheter balloon. The monitor 104 may be made of compliant or non-compliant material. As illustrated, the monitor 104 is generally in the shape of a torus, although it may be any suitable shape. The monitor 104 is filled with a fluid and is connected to the distal end of a monitor lumen 106. The monitor lumen 106 at is proximal end communicates with a hydraulic pressure gauge 108, shown schematically in FIG. 8. The gauge may be formed as part of the catheter itself, e.g., located in the manifold, or it may be an external device. The gauge is capable of measuring the pressure on the monitor 104. The gauge may be analog or digital and may be calibrated to give readouts specific to the desired application. For example, it may display pressure or a calibrated force.

When the catheter is advanced and comes into contact with tissue 110, the monitor 104 is compressed, increasing the fluid pressure in the monitor. The gauge detects this increase in pressure, identifying to the operator that the catheter has come into contact with tissue. The gauge may also be used to determine the amount of force with which the catheter is being pressed up against the tissue. In this way, the operator can determine the appropriate time to stop advancement of the catheter, extend the needle, and perform the injection. It helps insure that the needle is not extended prior to the catheter reaching the tissue, and it also helps insure that the operator does not continue to apply advancement force to the catheter after it has reached the target position. The application of too much force can damage tissue, as can incorrect needle deployment.

The inflation fluid for the monitor 104 may be a radiopaque contrast agent. In this way the operator can also visualize contact through a change of shape of the monitor 104 due to compression.

It will be appreciated that an injection catheter in accordance with the invention may be used to deliver any pharmaceutically acceptable therapeutic agent, such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl) ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor αa, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻ CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Although certain embodiments of the present invention have been illustrated and described in detail, it should be understood that various changes, substitutions, and alterations may be made within the scope of the invention. The invention is intended to cover various modifications and equivalent arrangements. Other examples are readily ascertainable from the above description by one skilled in the art and may be made without departing from the spirit and scope of the present invention, which is defined by the following claims.

What is claimed is:

1. An injection catheter comprising:
an elongated shaft having a distal end and a proximal end and a first lumen extending therebetween;
a needle with a proximal end and a distal end and a needle lumen extending therebetween, the needle disposed within the first lumen of the elongated shaft and extending from a proximal end of the catheter to a distal end of the catheter; and
a flexible sleeve comprising a plurality of rings and at least one connector for connecting at least two of the plurality of rings.

2. The injection catheter of claim 1 wherein the connector connects at least three of the rings of the flexible sleeve.

3. The injection catheter of claim 1 wherein the connector connects all of the rings of the flexible sleeve.

4. The injection catheter of claim 1 wherein the connector extends in a direction parallel to a longitudinal axis of the sleeve.

5. The injection catheter of claim 4 wherein the connector connects at least three of the rings of the flexible sleeve.

6. The injection catheter of claim 4 wherein the connector connects all of the rings of the flexible sleeve.

7. The injection catheter of claim 1 wherein the connector extends in a helical direction with respect to a longitudinal axis of the sleeve.

8. The injection catheter of claim 7 wherein the connector connects at least three of the rings of the flexible sleeve.

9. The injection catheter of claim 7 wherein the connector connects all of the rings of the flexible sleeve.

10. A method of using an injection catheter comprising the steps of:
(i) providing an injection catheter comprising:
(a) an elongated shaft having a distal end and a proximal end and a first lumen extending therebetween;
(b) a needle with a proximal end and a distal end and a needle lumen extending therebetween, the needle disposed within the first lumen of the elongated shaft and extending from a proximal end of the catheter to a distal end of the catheter; and
(c) a flexible sleeve; and
(ii) bending the catheter along a curve, with the flexible sleeve compressing along the inside of the curve and the flexible sleeve elongating along the outside of the curve.

11. An injection catheter comprising:
an elongated shaft having a distal end and a proximal end and a first lumen extending therebetween;
a needle with a proximal end and a distal end and a needle lumen extending therebetween, the needle disposed within the first lumen of the elongated shaft and extending from a proximal end of the catheter to a distal end of the catheter;
a stop surface fixed with respect to the elongated shaft;
a needle stop fixed with respect to the needle; and
a distal tissue contact surface, wherein the position of the distal tissue contact surface with respect to the stop surface is adjustable.

12. The injection catheter of claim 11 wherein the catheter further comprises an adjustable hood, and wherein the distal tissue contact surface is located on the adjustable hood.

13. The injection catheter of claim 12 wherein the adjustable hood is adjustable by a threaded engagement.

14. A method of using an injection catheter comprising the steps of:
(i) providing an injection catheter comprising:
(a) an elongated shaft having a distal end and a proximal end and a first lumen extending therebetween;
(b) a needle with a proximal end and a distal end and a needle lumen extending therebetween, the needle disposed within the first lumen of the elongated shaft and extending from a proximal end of the catheter to a distal end of the catheter;
(c) a stop surface fixed with respect to the elongated shaft;
(d) a needle stop fixed with respect to the needle; and
(e) a distal tissue contact surface; and
(ii) adjusting the position of the distal tissue contact surface with respect to the stop surface.

15. The method of claim 14 wherein the catheter further comprises an adjustable hood, and wherein the distal tissue contact surface is located on the adjustable hood.

16. The method of claim 15 wherein the adjustable hood is adjustable by a threaded engagement.

17. A catheter comprising:
an elongated shaft having a distal end and a proximal end and a first lumen extending therebetween;
a fluid pressure contact monitor located at the distal end of the elongated shaft; and
a monitor lumen having a proximal end and a distal end, the distal end of the monitor lumen connected to the fluid pressure contact monitor and the proximal end of the monitor lumen adapted to be connected to a gauge.

18. The catheter of claim 17 wherein the fluid pressure contact monitor comprises an inflatable balloon.

19. A method of using a catheter comprising the steps of:
(i) providing an catheter comprising:
(a) an elongated shaft having a distal end and a proximal end and a first lumen extending therebetween;
(b) a fluid pressure contact monitor located at the distal end of the elongated shaft; and
(c) a monitor lumen having a proximal end and a distal end, the distal end of the monitor lumen connected to the fluid pressure contact monitor and the proximal end of the monitor lumen adapted to be connected to a gauge;
(ii) advancing the catheter within a body lumen;
(iii) detecting the pressure in the fluid pressure contact monitor.

20. The method of claim 19 wherein the fluid pressure contact monitor comprises an inflatable balloon.
